(19) United States
(12) Patent Application Publication
(10) Pub. No.: US 2015/0325139 A1
(43) Pub. Date: Nov. 12, 2015

(54) APPARATUS AND METHOD FOR SUPPORTING REHABILITATION OF BRAIN-DAMAGED PATIENT

(71) Applicant: Samsung Electronics Co., Ltd., Suwon-si (KR)

(72) Inventors: Hyo A KANG, Seoul (KR); Hye Jin KAM, Seongnam-si (KR)

(73) Assignee: Samsung Electronics Co., Ltd., Suwon-si (KR)

(21) Appl. No.: 14/693,703

(22) Filed: Apr. 22, 2015

(30) Foreign Application Priority Data
May 9, 2014 (KR) 10-2014-0055891

(51) Int. Cl. G09B 19/00; G09B 5/00; A61B 5/00

(52) U.S. Cl. CPC G09B 19/00; A61B 5/4064; A61B 5/7271; G09B 5/00

(57) ABSTRACT

An apparatus that supports a rehabilitation of a brain-damaged patient, including a patient condition analyzer configured to generate a patient condition scenario about a brain condition of a patient based on brain-related information of the patient, a network model determiner configured to determine a network model to be applied to the patient using the patient condition scenario, and a rehabilitation model generator configured to generate a rehabilitation model to be applied to the patient based on the determined network model.

### CROSS-REFERENCE TO RELATED APPLICATION(S)

This application claims the benefit under 35 USC 119(a) of Korean Patent Application No. 10-2014-0055891, filed on May 9, 2014, in the Korean Intellectual Property Office, the entire disclosure of which is incorporated herein by reference for all purposes.

### BACKGROUND

1. Field

The following description relates to an apparatus and method to support a rehabilitation of a brain-damaged patient, and more particularly, to a technique for supporting an activation of a brain area capable of substituting or compensating for a damaged brain area.

2. Description of the Related Art

Adults who have suffered a stroke may have difficulty recovering because brain cells do not reproduce. Since early 2000's, numerous researches have suggested that an undamaged brain area may substitute a function of a damaged brain area so that the brain damage may recover. A substitution of a function of an undamaged brain area for a function of a damaged brain area is referred to as brain plasticity. Efforts have been made to study how to apply brain plasticity to the rehabilitation of patients who have suffered a stroke, or to those with atopic dermatitis, which can cause behavior/cognitive impairment.

More recently, it has been determined that electronic/magnetic/visual stimulation on a damaged brain area may help reinforce the effects of rehabilitation. Currently, there are numerous research efforts directed toward the question of how to stimulate a substitute brain area, however, conventional methods to determine the substitute brain area are often still implemented. These conventional methods are based on the well-known functions of brain areas, or on empirical bases, and may fail to take fully into consideration their association or relevance to a rehabilitation effort.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, an apparatus that supports a rehabilitation of a brain-damaged patient including a patient condition analyzer configured to generate a patient condition scenario about a brain condition of a patient based on brain-related information of the patient, a network model determiner configured to determine a network model to be applied to the patient using the patient condition scenario, and a rehabilitation model generator configured to generate a rehabilitation model to be applied to the patient based on the determined network model is provided

The rehabilitation model generator is further configured to generate the rehabilitation model to be applied to the patient based on a rehabilitation efficiency mode.

The patient condition analyzer is further configured to extract a feature value for a primary feature by analyzing the brain-related information of the patient, and to generate the patient condition scenario by applying an analytical technique to the extracted feature value.

The patient condition analyzer is further configured to extract a feature value for a primary feature by analyzing the brain-related information of the patient, and to generate the patient condition scenario by using a patient information item that is acquired based on similarity between a change over time of the extracted feature value and a brain-related disease progression information of the patient stored in a patient information database (DB).

The network model determiner is further configured to determine at least one of a currently damaged brain area and an anticipated area of further brain damage by using the patient condition scenario, to extract one or more sub network models associated with the determined area from the brain area network model, and to determine at least one of the one or more extracted sub network models as a network model to be applied to the patient.

The network model determiner is further configured to determine a functional brain change of the determine area using the patient condition scenario, and to assign weight values to the one or more extracted sub network models based on the determined functional brain change, and, based on the assigned weight values, to determine at least one of the one or more extracted sub network models as a network model to be applied to the patient.

The rehabilitation model generator is further configured to determine a substitute area or a compensating area for a function of the determine area using the determined network model, and to generate a rehabilitation model for rehabilitation of the determined area based on the rehabilitation efficiency model.

The rehabilitation model generator is further configured to determine a rehabilitation program based on one or more information items in knowledge information and rehabilitation information of patients who are in condition similar to the patient condition scenario, to predict effects that contain a degree of functional recovery expected by applying the determined rehabilitation program, and to generate a rehabilitation model including the determined rehabilitation program and the predicted effects.

The apparatus further includes a rehabilitation model provider configured to provide the generated rehabilitation model to a user

In another general aspect, a method of supporting a rehabilitation of a brain-damaged patient including generating a patient condition scenario about a brain condition of a patient based on brain-related information of the patient, determining a network model to be applied to the patient in a brain area network model based on the patient condition scenario and generating a rehabilitation model to be applied to the patient based on the determined network model is provided.

The rehabilitation model is further generated to be applied to the patient based on a rehabilitation efficiency model.

The generating of a patient condition scenario includes extracting a feature value for one or more primary feature by analyzing the brain-related information of the patient, and generating the patient condition scenario by applying a predetermined analytic technique to the extracted feature value.

The generating of a patient condition scenario includes extracting a feature value for one or more primary features by analyzing the brain-related information of the patient, and generating the patient condition scenario by using one or more patient information items that are acquired based on a change over time of the extracted feature value and similarity between patient's brain-related disease progression information stored in the patient information database (DB).

The determining of a network model includes determining at least one of a currently damaged brain area and an anticipated area of further brain damage by using the patient condition scenario, extracting one or more sub networks associated with the determined area from the brain area network model, and determining at least one of the one or more extracted sub network models as a network model to be applied to the patient.

The determining of a network model further may include determining a functional brain change of the determined area using the patient condition scenario, and wherein the determining as to a network model to be applied to the patient includes assigning a weight value to the one or more extracted sub networks based on the functional brain change of the determined area, and based on the assigned weight value, determining at least one of the one or more extracted sub network models as a network model to be applied to the patient.

The generating of a rehabilitation model includes determining a substitute area or a compensating area for a function of the determined region by using the determined network model, and generating a rehabilitation model for rehabilitation of the determined region based on the rehabilitation efficiency model.

The generating of a rehabilitation model includes determining a rehabilitation program based on one or more information items in knowledge information and rehabilitation information of patients who are in condition similar to the patient condition scenario, predicting effects that contain a degree of functional recovery expected by applying the determined rehabilitation program, and generating a rehabilitation model including the determined rehabilitation program and the predicted effects.

The method may further include providing the generated rehabilitation model to a user.

In yet another general aspect, there is provided an apparatus for supporting a rehabilitation of a brain-damaged patient including patient information database (DB) configured to store at least one of disease information and rehabilitation information of brain-damaged patients, a knowledge information DB configured to store brain-related knowledge information, and a network model generator configured to generate a brain area network model by analyzing association between brain areas based on information stored in either the patient information DB or the knowledge information DB

The network model generator is further configured to generate the brain area network model by determining a substitute area or a compensating area of each brain area and constructing a network between a specific brain area and the determined area.

The network model generator is further configured to determine one or more areas capable of substituting or compensating for one or more functions of each brain area, to generate one or more sub network models for the one or more functions of each brain area, and To generate the brain area network model including the one or more generated sub network model

The apparatus may further include a rehabilitation efficiency model generator configured to generate a rehabilitation efficiency model by analyzing effects of a rehabilitation program for each brain area based on one or more information items stored in the patient information DB and the knowledge information DB.

The effects of the rehabilitation program include one or more of a degree of functional recovery expected by applying the rehabilitation program, a participation of the patient about the rehabilitation program a satisfaction of the patient about the rehabilitation program, a convenience of the rehabilitation program, and a psychological and a physical stress that the patient suffers during an application of the rehabilitation program.

The apparatus may further include a patient information collector configured to collect the patient's rehabilitation information obtained by applying the rehabilitation model to the patient, and to store the collected rehabilitation information in the patient information DB.

In yet another general aspect, an apparatus for supporting a rehabilitation of a brain-damaged patient including a modeler configured to generate a brain area network model including association information of brain areas and a rehabilitation model for each brain area by analyzing eat least one of brain-related patient information and brain-related knowledge information, and an applier configured to, in response to receipt of brain-related information of a new patient, generate a rehabilitation model to be applied to the new patient, by analyzing the brain-related information, the brain area network model, and the rehabilitation model all together is provided

The modeler is further configured to generate one or more sub networks for one or more functions of each brain area, and generate the brain area network model including the one or more generated sub networks.

The modeler is further configured to predict effects of the rehabilitation program, which includes a degree of functional recovery expected by applying the rehabilitation program to each brain area, and generate the rehabilitation efficiency model including the predicted effects of the rehabilitation program.

The applier is further configured to collect the patient's rehabilitation information obtained by applying the rehabilitation model to the patient, and transmit a feedback on the collected rehabilitation information to the modeler.

Other features and aspects may be apparent from the following detailed description, the drawings, and the claims.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram illustrating an apparatus to support a rehabilitation of a brain-damaged patient, according to an embodiment.

FIG. 2 is a block diagram illustrating an apparatus to support a rehabilitation of a brain-damaged patient, according to another embodiment.

FIGS. 3A to 3C are diagrams illustrating brain activity and a brain area network model.

FIG. 4 is a block diagram illustrating an apparatus to support a rehabilitation of a brain-damaged patient, according to another embodiment.

FIGS. 5A to 5C are diagrams illustrating a rehabilitation model to be applied to a brain-damaged patient.

FIG. 6 is a flowchart illustrating a method to support a rehabilitation of a brain-damaged patient, according to an embodiment.

FIG. 7 is a flowchart illustrating a method to support a rehabilitation of a brain-damaged patient, according to another embodiment.

FIG. 8 is a flowchart illustrating a method to support a rehabilitation of a brain-damaged patient, according to a further embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. For example, the sequences of operations described herein are merely examples, and are not limited to those set forth herein, but may be changed as will be apparent to one of ordinary skill in the art, with the exception of operations necessarily occurring in a certain order. Also, descriptions of well-known functions and constructions may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The terminology used herein is for the purpose of describing particular embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Hereinafter, an apparatus and method to support rehabilitation of a brain-damaged patient are described with reference to drawings.

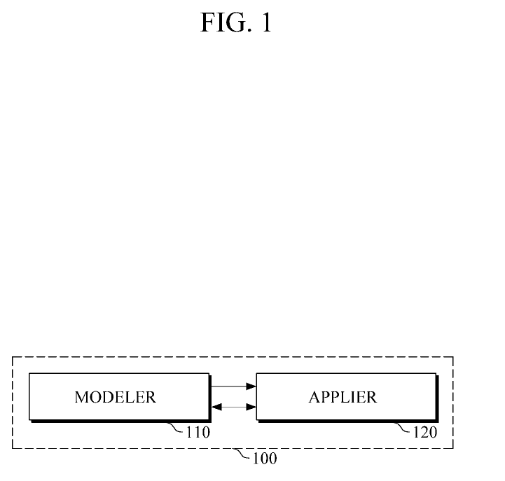

FIG. 1 is a block diagram illustrating an apparatus to support a rehabilitation of a brain-damaged patient, according to an embodiment.

Referring to FIG. 1, an apparatus 100, which supports the rehabilitation of the brain-damaged patient, includes a modeler 110 and an applier 120.

The modeler 110 generates a brain area network model and a rehabilitation efficiency model for each brain area by analyzing brain-related patient information and brain-related knowledge information. In accordance with one illustrative configuration, the modeler 110 is an extensive predictive processor that is configured to bring predictive intelligence to decisions to be made.

The brain-related patient information includes various types of information, including information from a plurality of brain damaged patients. For example, the brain-related patient information includes, but it is not limited to, a structural computed tomography (CT) image, such as an X-ray image, a CT image, a Magnetic Resonance Imaging (MRI) image, a functional brain CT image, such as a functional Magnetic Resonance Imaging (fMRI) image and a Positron Emission Tomography (PET) image, or multiple consecutive images. The brain CT image may be an image captured more than twice for one or more brain areas. In addition, the patient information may include a text extracted from an image, as well as a graph indicative of an association between brain areas.

The information monitored when applying a rehabilitation model to a patient may include various types of rehabilitation effectiveness data, such as function recovery data, the level of a patient's participation, the convenience of the model, the psychological and physical pain that a patient may suffer during rehabilitation, and the level of patient satisfaction with the results of the rehabilitation program.

In addition, the patient information may include an Electronic Medical Record (EMR) managed by a hospital for each patient, which may have therein, a gender, an age, a disease history, a family history, any lab results attained from a general clinical environment, and any results on functional examinations of memory, of linguistic capability, and of physical exercise.

In an embodiment, the knowledge information may include guideline information used in rehabilitation; association information of brain areas, which is extracted from documents using machine learning or data mining; rehabilitation information; and information on types and methods of rehabilitation.

By analyzing various types of patient information, which collected from a plurality of patients, and various types of knowledge information generated by medical experts, the modeler 110 generates a brain area network model by determining a substitute or a compensating area of each brain area and then constructing a network associating each brain area and a determined substitute/compensating area.

A brain area network model indicates an association of each brain area with an area that substitutes or compensates for a function of a corresponding brain area. In an embodiment, the substitute area refers to a brain area that performs a function that is identical or substantially identical to a function of a specific brain area, where the function of the specific brain area has changed or has been damaged. In addition, the compensating area refers to a brain area that performs a function which is not identical to, but compensates for, a changed or damaged function of the specific brain area.

That is, with respect to the rehabilitation of a specific brain activity or function regarding linguistics, thinking skills, movement, memory, and the like, the brain area network model is a functional association map that helps a damaged function to be recovered or compensated for through a natural process of healing or through rehabilitation, when a specific brain area is damaged.

For example, the modeler 110 generates a graph-typed brain area network model by mapping documents and expert knowledge, which are recorded in text on a location in a brain structure of a patient. In the alternative, the modeler 110 generates the graph-typed brain area network model by applying various mathematical modeling techniques or statistics-based analytic techniques, such as pattern recognition and machine learning algorithms, to various types of patient information associated with patient's existing clinical information and function recovery led by rehabilitation. In one illustrative example, the patient may be a person or an animal.

By analyzing various types of collected information, the modeler 110 is configured to predict certain effects of a rehabilitation program to be applied to a specific brain area, and generates a rehabilitation efficiency model that includes the predicted effects. For example, after determining the effects of a type of a rehabilitation program and a method to execute of the rehabilitation program for each patient in a set of patient information or of knowledge information, the modeler 110 then generates a rehabilitation model by applying any of various techniques, such as mathematical modeling techniques, statistics-based analytical techniques, and machine learning techniques.

In an embodiment, the rehabilitation program includes at least one of exercise therapy, physical therapy, medicine therapy, stimulation therapy by stimulating a specific brain area, and psychiatric counseling. In addition, effects of the rehabilitation program may include the degree of function recovery achieved due to the application of the rehabilitation program, a participation of a patient in the rehabilitation program, a convenience of the rehabilitation program, any psychological and physical stresses that the patient suffered during application of the rehabilitation program, and a level of patient satisfaction associated with an expected result.

Once brain-related information of a new patient is inputted, the applier 120 analyzes the brain-related information. Results of the analysis of the brain-related information are combined with the brain area network model and the rehabilitation efficiency model for each brain area produced by the modeler 110 to generate a rehabilitation model optimized for the patient. The rehabilitation model is optimized for the patient and includes a rehabilitation program and predicted effects thereof, which may further include a degree of function recovery expected by applying the rehabilitation program.

The rehabilitation program, based on the rehabilitation model, includes physical therapy of various types, intensity, timing, and duration; medication therapy of various types and amounts needed in increasing rehabilitation efficacy, medication timing, and medication duration; stimulation therapy of brain areas using electric and magnetic forces, stimulation intensity, stimulation timing, and stimulation duration; physical therapy of various exercise types, intensity, timing, and duration; and psychiatric therapy of various types of psychiatric consultation programs, and duration.

The applier 120 provides the rehabilitation model generated for a patient to a user including the patient or a medical staff member, including a medical doctor.

In addition, the applier 120 feeds a result of the application of the rehabilitation model, which is input by the patient or the medical staff member, back into the modeler 110 to reflect the result in generating a rehabilitation model. The information fed back by the applier 120 includes effects expected by applying the rehabilitation model.

The modeler 110 manages the information fed back by the applier 120 as patient information, and utilizes the information to generate the brain area network model and the rehabilitation efficiency model.

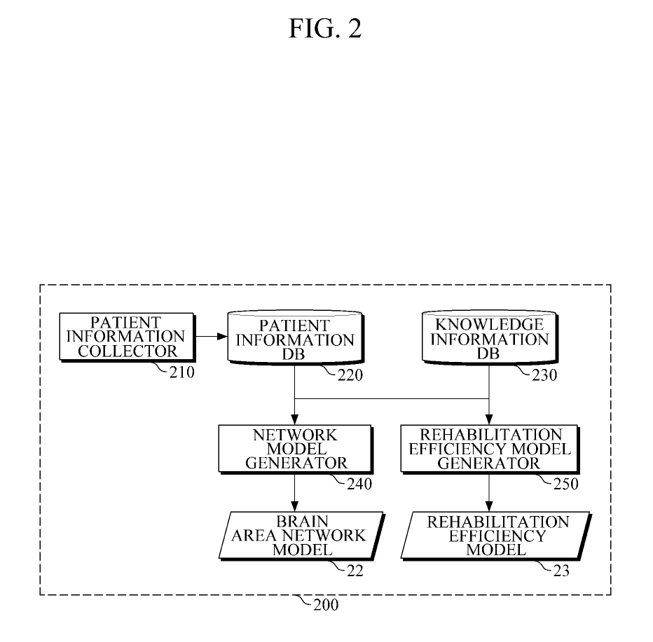

FIG. 2 is a block diagram illustrating an example of an apparatus to support a rehabilitation of a brain-damaged patient, according to another embodiment.

Referring to FIG. 2, an apparatus 200 to support rehabilitation is an illustrative example of the modeler 110 shown in FIG. 1.

In FIG. 2, the apparatus 200 includes a patient information collector 210, a patient information database (DB) 220, a knowledge information DB 230, a network model generator 240, and a rehabilitation efficiency model generator 250.

The patient information collector 210 collects rehabilitation information, such as effects expected by applying a rehabilitation model optimized for a patient, and stores the collected rehabilitation information in the patient information DB 220 for management.

The patient information DB 220 stores and manages patient information that include disease information of the patient and the rehabilitation information collected at the patient information collector 210. The patient information may include a structural or functional brain CT image, multiple-layered consecutive images, a text extracted from the image, a graph-typed association information of brain areas, information on the degree of function recovery expected by applying a rehabilitation program, and an Electronic Medical Record (EMR).

The knowledge information DB 230 stores and manages brain-related knowledge information, which is extracted from various sources, such as documents and research papers, about the brain. The brain-related knowledge information includes well-known association information of brain areas, guideline for rehabilitation treatment, and well-known effects of a rehabilitation program.

With reference to various types of brain-related information including the patient information and the brain-related knowledge information stored in the patient information DB 220 and the knowledge information DB 230, respectively, the network model generator 240 analyzes association between brain areas and generates a brain area network model using the analytical result.

For example, if the network model generator 240 determines that a specific brain area is damaged, the network model generator 240 generates a brain area network model 22 by determining a brain area capable of substituting or compensating for the specific brain area and then constructing a network between the specific brain a and the determined substitute/compensating brain area.

The network model generator 240 determines one or more areas capable of substituting/compensating for one or more functions of the specific brain area, and generates one or more sub network models for one or more functions of the specific brain area. In addition, the network model generator 230 generates the entire brain area network model 22, which includes each generated sub network model. In one illustrative example, the brain area network model 22 includes one or more sub networks for each brain area.

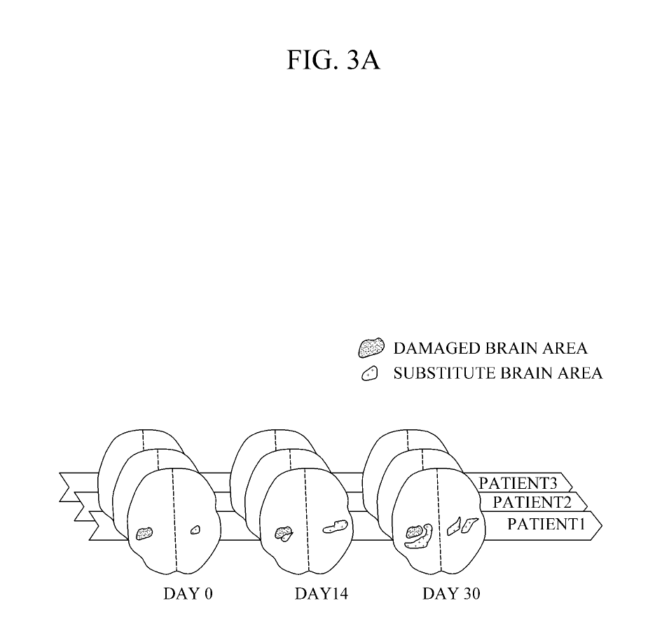

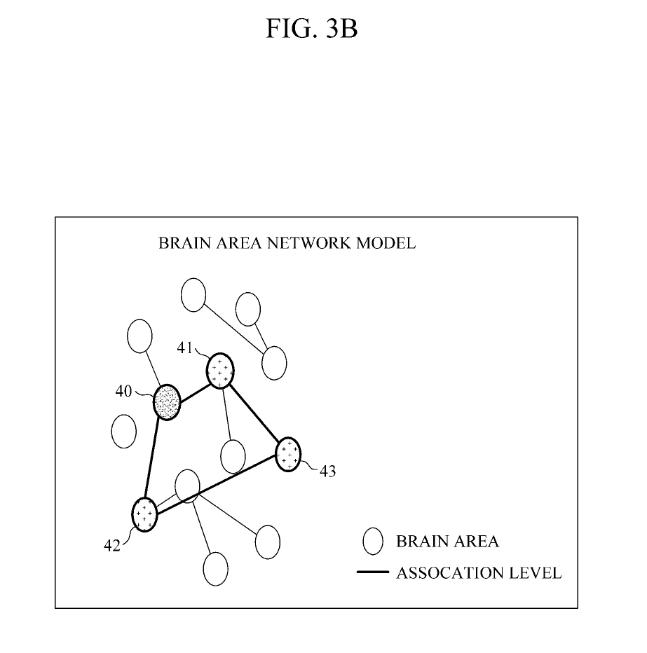

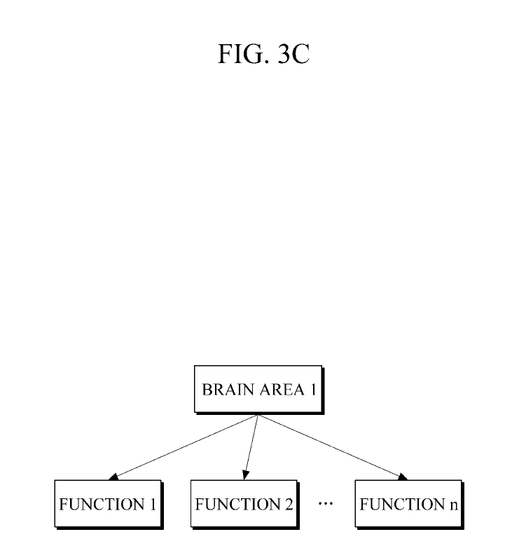

FIGS. 3A to 3C are diagrams illustrating brain activity and a brain area network model, in accordance with an embodiment.

FIG. 3A is a diagram illustrating brain activity image data of a brain-damaged patient, which are collected, chronicled and stored in the patient information DB 220.

Referring to FIG. 3A, a brain-damaged area and a substitute/compensating area for the brain-damaged area may be determined using the brain activity image data of Patient 1. As time lapses after a rehabilitation program applied to the substitute/compensating area of Patient 1, the substitute/compensating area may be found to become more active.

FIGS. 3B and 3C are diagrams illustrating a brain area network model, in accordance with an embodiment.

Referring to FIGS. 3B and 3C, the network model generator 240 determines substitute/compensating areas 41, 42 and 43 for a damaged brain area 40, as shown in FIG. 3B, by analyzing image data of a plurality of patients stored in the patient information DB 220, and generates a network model 22 by indicating an association between the substitute/compensating areas 41, 42, 43 and the damaged brain area 40. For example, in the case where the damaged brain area 40 is the left motor cortex, the substitute/compensating areas 41, 42 and 43 are the right motor cortex, the left sensory cortex, and the right sensory cortex, respectively.

As shown in FIG. 3B, the network model generator 240 a sub network by determining a substitute/compensating area capable of substituting or compensating for each function of a specific brain area. In this regard, the brain area network model 22 generated by the network model generator 240 includes one or more sub networks.

Referring back to FIG. 2, the rehabilitation efficiency model generator 250 analyzes a rehabilitation program's effects on each brain area by using rehabilitation-related information that are collected from various kinds of information stored in the patient information DB 220 or in the knowledge information DB 230. Furthermore, the rehabilitation efficiency model generator 250 may further analyze the rehabilitation program's effects based on the effects of a rehabilitation model that has been actually applied to patients or based on the experiences of experts. Subsequently, the rehabilitation efficiency model generator 250 generates the rehabilitation efficiency model 23 based on the analytical result.

In an embodiment, in a case where a specific brain area is damaged, the rehabilitation efficiency model generator 250 calculates rehabilitation efficiency as a numerical value, such as about 50% of function recovery compared to normalcy and about 70% of satisfaction of a patient, with respect to a substitute area or a compensating area of the damaged specific brain area. In addition, the rehabilitation efficiency model generator 250 generates a rehabilitation efficiency model 23 including each rehabilitation program's efficiency calculated for each brain area.

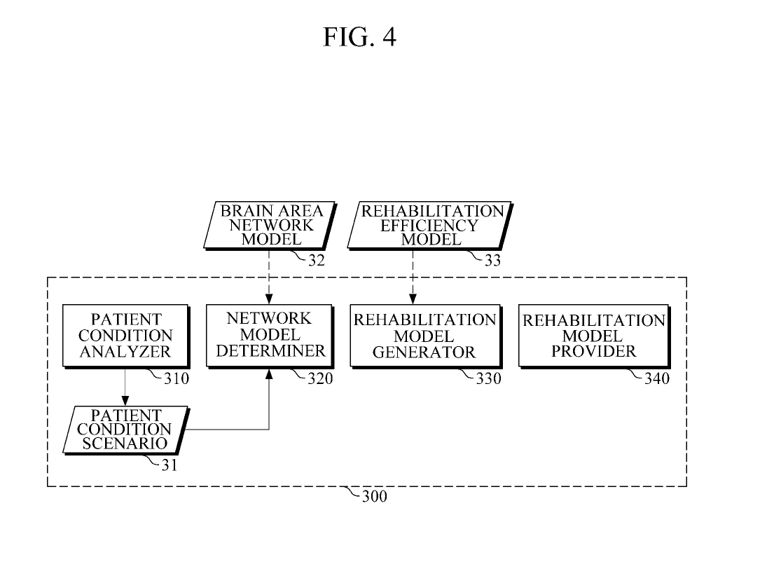

FIG. 4 is a block diagram illustrating an apparatus to support a rehabilitation of brain-damaged patient, according to another embodiment.

Referring to FIG. 4, an apparatus 300 to support rehabilitation is shown in FIG. 4.

In FIG. 4, the apparatus 300 includes a patient condition analyzer 310, a network model determiner 320, a rehabilitation model generator 330, and a rehabilitation model provider 340.

Upon receiving brain-related information of a new patient, the patient condition analyzer 310 analyzes the received brain-related information and generates a patient condition scenario 31 about a brain condition of the new patient.

In an embodiment, the patient condition scenario 31 includes structural brain change information and functional brain change prediction information of the patient. In accordance with an example, the structural brain change information includes one or more of the currently damaged brain area(s), a structural brain change, and structural brain change trends. In addition, the functional brain change prediction information includes one or more of the following in relation to the structural brain change trends, including, but not limited to, a speed, a direction, and any of various characteristics.

The patient condition analyzer 310 detects from the structural and functional brain changes, an Area of Interest (AOI) or a patterns, by analyzing received brain-related information of the patient, or extracts feature values for one or more primary features and then generates a patient condition scenario 31 based on the extracted feature values.

The primary feature refers to a feature that effectively reflects characteristics of a brain-related disease of the patient. For example, a primary feature includes measurements of a disease from lab work on amyloid beta level/blood pressure level, of vascular compliance, a result of a functional examination on memory, on linguistic capability, on mobility, brain volume measured from an image scanned by a brain imaging device, a thickness of each brain area a shape of the brain, a distribution of brain activity when a specific function is served, a change in brain activity with time, a vascular distribution, and a vascular thickness.

The patient condition analyzer 310 uses the patients' measurement data (e.g., raw data) stored in the patient information DB 220 as feature values in a manner in which such usage adequately corresponds to the respective primary features. Alternatively, the patient condition analyzer 310 calculates a ratio using a sum, an average, a median, a maximum/minimum, a variance, a standard deviation, the number of outliners, a value more or less than a reference value, or two or more measurement values of the data, and subsequently uses the calculated ratio as a primary feature.

According to an embodiment, the patient condition analyzer 310 generates the patient condition scenario 31 by applying a predetermined analytical technique to the extracted primary features. For example, the patient condition analyzer 310 generates the patient condition scenario 31 by applying a pattern recognition technique, a machine learning algorithm technique, a general mathematical modeling technique, and a time-series data analytical technique, which are described above, to a change over time of each extracted primary feature.

According to another embodiment, the patient condition analyzer 310 generates the patient condition scenario 31 based on a similarity between a change over time of each extracted feature and patients' disease progress information pre-collected in the patient information DB 220. The patient condition analyzer 310 acquires patient information of the patient, who is in a condition most similar to the change over time of each extracted feature. The patient condition analyzer 310 acquires the patient information based on a general similarity measure designed to measure the similarity between different types of information. Then, by taking into consideration acquired disease progression information of the patient, the patient condition analyzer 310 generates the patient condition scenario 31 which may, for example, indicate a direction of a new patient's disease progress.

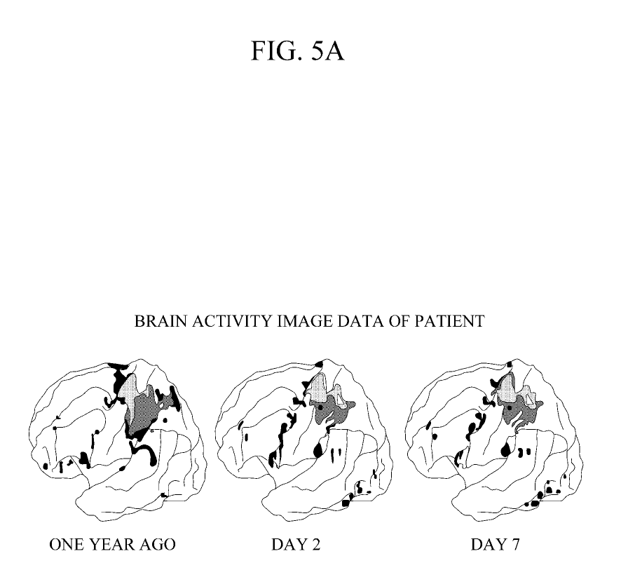

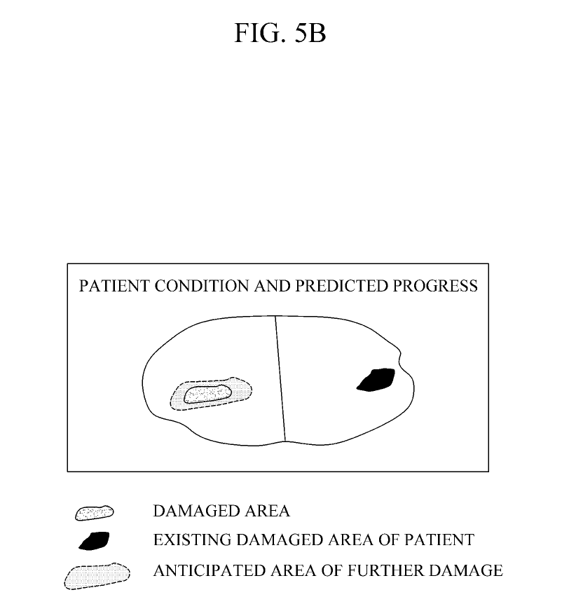

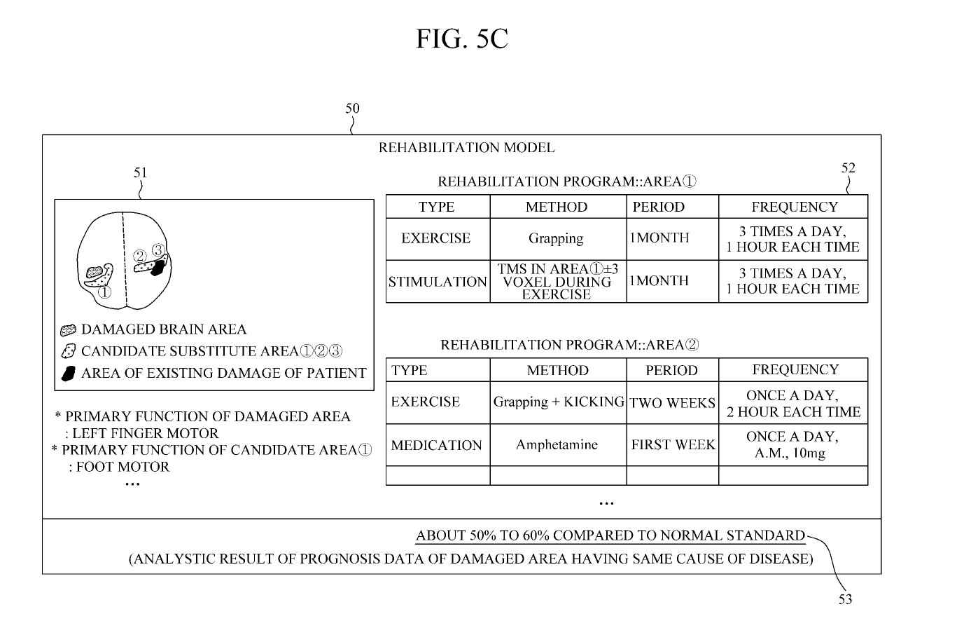

FIGS. 5A to 5C are diagrams illustrating a rehabilitation model to be applied a brain damaged patient.

Referring to FIG. 5A, the figure is a diagram illustrating brain activity image data of a new patient; that is, a diagram illustrating brain image data captured over time. FIG. 5B is a diagram illustrating the patient's condition and predicted disease progress showing an anticipated area of further damage, which is a result of an analysis performed by the patient condition analyzer 310 using the image data shown in FIG. 5A. Specifically, FIG. 5B shows the currently damaged brain area, the pre-existing damaged brain area, and an anticipated area of further brain-damage.

By analyzing a consolidation of a patient condition scenario generated by a patient condition analyzer 310, as shown in FIG. 4, a brain area network model 32 and a rehabilitation efficiency model 33, the network model determiner 320 determines a network model to be applied to the patient.

By using the patient condition scenario 31, the network model determiner 320 determines the currently damaged brain area and determines an area which is anticipated to include further brain-damage. Next, the network model determiner 320 extracts one or more sub network models associated with the determined areas from the brain area network model 32. In addition, the network model determiner 320 determines a network model appropriate for the patient that is selected from among the extracted sub network models.

The network model determiner 320 further determines a change in brain function between the areas determined using the patient condition scenario 31. In addition, the network model determiner 320 determines a network model to be applied to the patient that is selected from among the extracted sub network models based on the determined change in brain function. The network model determiner 320 assigns a different weighed value to each extracted sub network model based on the determined change in brain function, and determines a network model to be applied to the patient based on weights assigned to the extracted sub network models.

In an example embodiment, a weighed value may be assigned to an extracted sub network model according to the degree of damage based on an expected change in the functioning of a damaged brain area. That is, referring to FIG. 3C, in a case where a damaged brain area of a patient is Brain Area 1 and expected changes in function are include damage to Function 1 and to Function 2, the greatest weighed value is assigned to a sub network for Function 2 because it is the function that is expected to be the most damaged. Accordingly, the sub network for Function 2 is determined as a network model to be applied to the patient.

Alternatively, a weighed value may be assigned according to the degree of recovery associated with a change in function of a damaged area. That is, referring to FIG. 3C, in a case where an expected change in function is further damage to Function 1 and Function 2, and where Function 2 is expected to be the most damaged, if an expected degree of recovery of Function 2 is less than that of Function 1, a greater weighed value is assigned to a sub network for Function 1. Accordingly the sub network for Function 1 may be determined as a network model to be applied to the patient.

Using the network model that has been chosen, the rehabilitation model generator 330 determines a substitute area, alternatively referred to as a compensating area, of a damaged brain area and then generates a rehabilitation model used for rehabilitating the determined substitute area, or compensating area, with additional reference to a rehabilitation efficiency model.

In an embodiment, the rehabilitation model generator 330 may determine a rehabilitation program based on information including rehabilitation information of patients who are in a condition similar to the patient condition scenario 31. In this scenario, the rehabilitation model generator 330 predicts the effects of the rehabilitation program, which includes a prediction about the degree of function recovery. In addition, the rehabilitation model generator 330 may generate a rehabilitation model that includes the determined rehabilitation program and the predicted effects in the rehabilitation model.

Referring to FIG. 5C, a rehabilitation model 50 generated for a patient may include an analysis on the condition of the patient's brain 51, a rehabilitation program 52 according to the condition of the brain, and the predicted effects of applying the rehabilitation program, or in other words, the degree of brain function recovery 53. Referring to FIG. 5C, three substitute/compensating areas \textcircled{1}, \textcircled{2}, and \textcircled{3} have been determined for the damaged brain areas of a patient. Corresponding rehabilitation programs for the respective substitute/compensating areas have also been presented.

The rehabilitation model provider 340 provides to a user a rehabilitation model generated by the rehabilitation model generator 330. In an embodiment, the user may be the subject patient, or may be a medical staff member, such as a physical therapist or a doctor in charge of the rehabilitation of the patient.

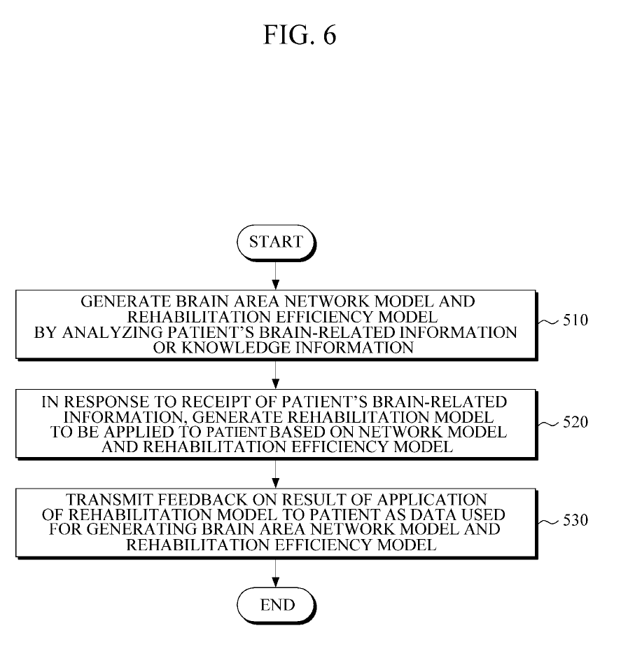

FIG. 6 is a flowchart illustrating a method for supporting a rehabilitation of brain-damaged patient according to an embodiment.

The embodiment of FIG. 6 is an example in which the apparatus 100 shown in FIG. 1 implements the method for supporting rehabilitation.

Referring to FIG. 6, the apparatus 100 generates a brain area network model and a rehabilitation efficiency model by analyzing multiple brain-damaged patients' brain-related patient information or other brain-related knowledge which has been collected from any of various sources including experts, various research papers, and the like, in operation 510.

As described above, the patient information and the other brain-related knowledge include various types of brain-related information such as existing analytical information about a patients' brain image data, information about the resultant effects of applying a rehabilitation program, and information regarding an association among brain areas. Thus, if a specific brain area is damaged, an association between the specific area and the area substituting or compensating for a function thereof may be obtained by utilizing and analyzing various types of brain-related information that have been collected in advance. Then, a brain area network model may be generated in any of various formats, such as a graph, to indicate the association between the specific damaged brain area and the corresponding substitute/compensating area thereof.

In addition, by using the rehabilitation information found within the patient information or information otherwise known about brain-damage, it is possible to predict certain effects, for example, to predict a degree of recovery expected of a particular function by applying a particular rehabilitation program to a specific brain area. It is also possible to generate a rehabilitation efficiency model in connection with the rehabilitation program for each brain area based on the predicted effects of the program.

In operation 520, in response to receipt of a brain-damaged patient's brain related information, for example, in response time-series brain CT images and multiple-layered image data, a damaged brain area of the patient and a substitute/compensating area thereof may be determined by analyzing the above information. A rehabilitation model to be applied to the patient may be generated based on the brain area network model and on the rehabilitation efficiency model generated in operation 510.

In operation 530, the rehabilitation model may be provided to a user that will apply the rehabilitation model to the patient. Various information, such as the effects of rehabilitation resulting from applying the rehabilitation model, may be fed back to operation 510.

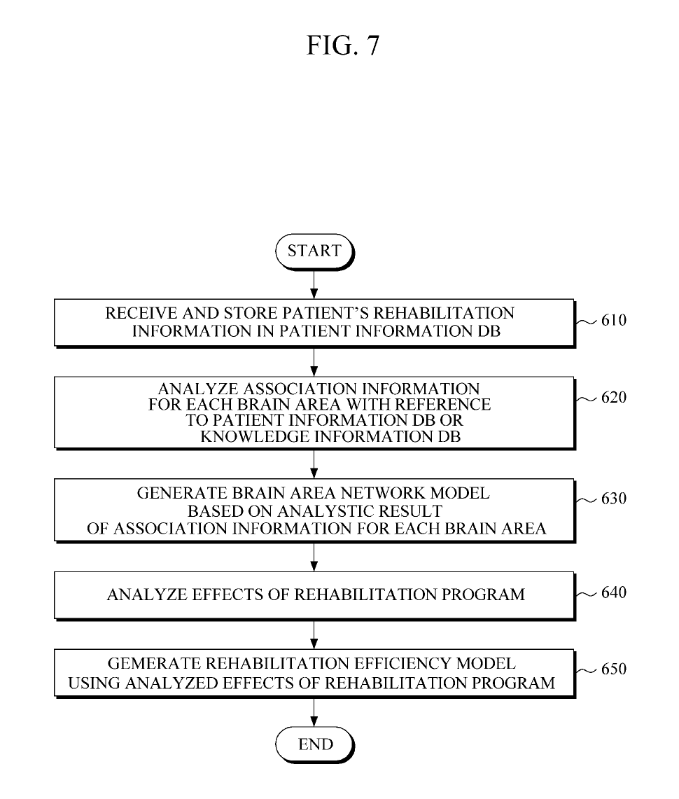

FIG. 7 is a flowchart illustrating a method for supporting a rehabilitation of brain-damaged patient according to another embodiment.

Referring to FIG. 7, the embodiment is an embodiment of a method for supporting rehabilitation which is implemented by the apparatus 200 shown in FIG. 2.

Referring to FIG. 7, in operation 610, in response to a feedback of rehabilitation information, such as the effectiveness of a rehabilitation model applied to a brain-damaged patient, the apparatus 200 receives the rehabilitation information and stores the rehabilitation information in the patient information DB. The patient information DB stores various patient information, such as disease information and rehabilitation information, of a plurality of patients.

In operation 620, the association between areas of the brain may be analyzed based on various types of brain-related information stored in the patient information DB or in a DB for other brain-related information. The association between brain areas refers to a relationship between one brain area and one or more other brain areas that are capable of substituting or compensating for one or more functions of a specific brain area that has been damaged.

After the association between brain areas is analyzed, a network model between brain areas is generated based on the analytical results in operation 630. An entire network model is generated by constructing a sub network for each function of a specific brain area.

In operation 640, in a case where one or more rehabilitation programs is applied to a specific brain area using various types of rehabilitation information stored in the patient information DB or the knowledge information DB, the effects of the rehabilitation program may be analyzed. In an embodiment, the rehabilitation effects is a numerical value indicative of the degree of recovery expected of a particular brain function by applying a particular rehabilitation program. In other embodiments, the rehabilitation effects relate to a level of participation from patients, to satisfaction experienced by patients, to stress the patients have suffered, and the like.

In operation 650, a rehabilitation efficiency model is generated using the results of the analysis on the effectiveness of each rehabilitation program.

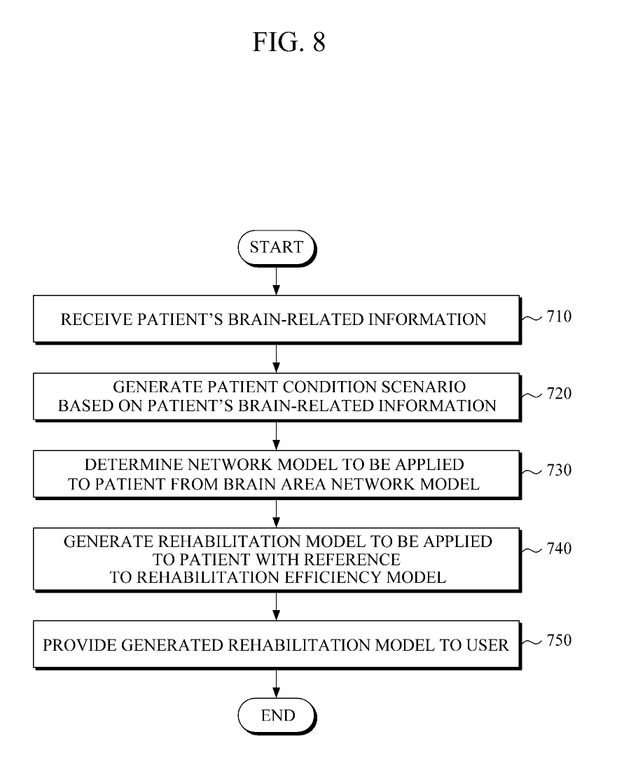

FIG. 8 is a flowchart illustrating a method for supporting a rehabilitation of brain-damaged patient according to another embodiment.

The embodiment of FIG. 8 is an embodiment of a method for supporting rehabilitation that is implemented by the apparatus 300 shown in FIG. 4.

Referring to FIG. 8, the apparatus 300 receives a brain-damaged patient's brain-related information, such as time-series brain image data in 710.

In operation 720, in response to receipt of the brain-related information of the new brain-damaged patient, a patient condition scenario about a brain condition of the patient is generated by analyzing the received information. The patient condition scenario may include reference to any of the following: the currently damaged brain area; to an area of a brain anticipated to have further brain-damage; to an area of existing brain damage; to a change in a brain function expected to occur due to a difference between the current brain-damage and the expected brain-damage; and to any of the degree, the speed, and the direction in functional change, and the like.

In operation 730, when the patient condition scenario of the patient is generated, a network model adequate for the patient is determined by analyzing the patient condition information and the existing brain area network model. In an embodiment, the network model adequate for the patient is a sub network related to a damaged brain area and a damaged function thereof in a brain area network model. In addition, a weighed values is assigned to each sub network model related to a function by considering a functional change of the currently damaged brain area or of an anticipated area of further damage; the degree of damage for each function; speed, direction, and the like in the functional change. One or more sub networks is determined based on the weighed values that are assigned.

In operation 740, once the network model for the patient has been determined, a rehabilitation model to be applied to the patient is generated based on previous rehabilitation models. In an embodiment, the rehabilitation model includes a different rehabilitation program for each substitute/compensating area associated with a function of a damaged brain area, in addition to effects of each rehabilitation program.

In operation 750, the generated rehabilitation model is provided to a user to apply the rehabilitation model to the patient. In this case, a rehabilitation expert, such as a doctor in charge, reviews and applies the rehabilitation model by modifying the same to be optimized for the patient if necessary.

It is possible to generate an efficient model for rehabilitating a brain-damaged patient by utilizing disease/rehabilitation information of the brain-damaged patient and various knowledge from experts and other reliable sources. In addition, it is possible to provide a rehabilitation model that has been optimized for a particular brain-damaged patient. In this regard, using the generated model reinforces the rehabilitation process. The various analyzers, determiners, providers, generators, scenarios, and models described above may be implemented using one or more hardware components or a combination of one or more hardware components.

A hardware component may be, for example, a physical device that physically performs one or more operations, but is not limited thereto. Examples of hardware components include processors, controllers, servers, mobile devices, and other similar structural components or devices.

It is to be understood that in an embodiment of the operations in FIGS. 6-8 are performed in the sequence and manner as shown although the order of some operations and the like may be changed without departing from the spirit and scope of the described configurations. In accordance with an illustrative example, a computer program embodied on a non-transitory computer-readable medium may also be provided, encoding instructions to perform at least the method described in FIGS. 6-8.

Program instructions to perform methods described in FIGS. 6-8, or one or more operations thereof, may be recorded, stored, or fixed in one or more non-transitory computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein may be easily construed by programmers skilled in the art to which the embodiments pertain based on and using the flow diagrams and block diagrams of the figures and their corresponding descriptions as provided herein.

A number of examples have been described above. Nevertheless, it should be understood that various modifications may be made. That is, while this disclosure includes specific examples, it will be apparent to one of ordinary skill in the art that various changes in form and details may be made in these examples without departing from the spirit and scope of the claims and their equivalents. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims. Therefore, the scope of the disclosure is defined not by the detailed description, but by the claims and their equivalents, and all variations within the scope of the claims and their equivalents are to be construed as being included in the disclosure.

What is claimed is:

1. An apparatus that supports a rehabilitation of a brain-damaged patient, the apparatus comprising:
a patient condition analyzer configured to generate a patient condition scenario about a brain condition of a patient based on brain-related information of the patient;
a network model determiner configured to determine a network model to be applied to the patient using the patient condition scenario; and
a rehabilitation model generator configured to generate a rehabilitation model to be applied to the patient based on the determined network model.

2. The apparatus of claim 1, wherein the rehabilitation model generator is further configured to generate the rehabilitation model to be applied to the patient based on a rehabilitation efficiency mode.

3. The apparatus of claim 1, wherein the patient condition analyzer is further configured to extract a feature value for a primary feature by analyzing the brain-related information of the patient, and to generate the patient condition scenario by applying an analytical technique to the extracted feature value.

4. The apparatus of claim 1, wherein the patient condition analyzer is further configured to extract a feature value for a primary feature by analyzing the brain-related information of the patient, and to generate the patient condition scenario by using a patient information item that is acquired based on similarity between a change over time of the extracted feature value and a brain-related disease progression information of the patient stored in a patient information database (DB).

5. The apparatus of claim 1, wherein the network model determiner is further configured to determine at least one of a currently damaged brain area and an anticipated area of further brain damage by using the patient condition scenario, to extract one or more sub network models associated with the determined area from the brain area network model, and to determine at least one of the one or more extracted sub network models as a network model to be applied to the patient.

6. The apparatus of claim 5, wherein the network model determiner is further configured to determine a functional brain change of the determine area using the patient condition scenario, and to assign weight values to the one or more extracted sub network models based on the determined functional brain change, and, based on the assigned weight values, to determine at least one of the one or more extracted sub network models as a network model to be applied to the patient.

7. The apparatus of claim 5, wherein the rehabilitation model generator is further configured to determine a substitute area or a compensating area for a function of the determine area using the determined network model, and to generate a rehabilitation model for rehabilitation of the determined area based on the rehabilitation efficiency model.

8. The apparatus of claim 1, wherein the rehabilitation model generator is further configured to determine a rehabilitation program based on one or more information items in knowledge information and rehabilitation information of patients who are in condition similar to the patient condition scenario, to predict effects that contain a degree of functional recovery expected by applying the determined rehabilitation program, and to generate a rehabilitation model including the determined rehabilitation program and the predicted effects.

9. The apparatus of claim 1, further comprising:
a rehabilitation model provider configured to provide the generated rehabilitation model to a user.

10. A method of supporting a rehabilitation of a brain-damaged patient, the method comprising:
generating a patient condition scenario about a brain condition of a patient based on brain-related information of the patient;
determining a network model to be applied to the patient based on the patient condition scenario; and
generating a rehabilitation model to be applied to the patient based on the determined network model.

11. The method of claim 10, wherein the rehabilitation model is further generated to be applied to the patient based on a rehabilitation efficiency model.

12. The method of claim 10, wherein the generating of a patient condition scenario comprises:
extracting a feature value for one or more primary feature by analyzing the brain-related information of the patient; and
generating the patient condition scenario by applying a predetermined analytic technique to the extracted feature value.

13. The method of claim 10, wherein the generating of a patient condition scenario comprises:
extracting a feature value for one or more primary features by analyzing the brain-related information of the patient; and
generating the patient condition scenario by using one or more patient information items that are acquired based on a change over time of the extracted feature value and similarity between patient's brain-related disease progression information stored in the patient information database (DB).

14. The method of claim 10, wherein the determining of a network model comprises:
determining at least one of a currently damaged brain area and an anticipated area of further brain damage by using the patient condition scenario;
extracting one or more sub networks associated with the determined area from the brain area network model; and
determining at least one of the one or more extracted sub network models as a network model to be applied to the patient.

15. The method of claim 14,
wherein the determining of a network model further comprises determining a functional brain change of the determined area using the patient condition scenario, and
wherein the determining as to a network model to be applied to the patient comprises:
assigning a weight value to the one or more extracted sub networks based on the functional brain change of the determined area; and
based on the assigned weight value, determining at least one of the one or more extracted sub network models as a network model to be applied to the patient.

16. The method of claim 14, wherein the generating of a rehabilitation model comprises:
determining a substitute area or a compensating area for a function of the determined region by using the determined network model; and
generating a rehabilitation model for rehabilitation of the determined region based on the rehabilitation efficiency model.

17. The method of claim 14, wherein the generating of a rehabilitation model comprises:
determining a rehabilitation program based on one or more information items in knowledge information and rehabilitation information of patients who are in condition similar to the patient condition scenario, predicting effects that contain a degree of functional recovery expected by applying the determined rehabilitation program, and generating a rehabilitation model including the determined rehabilitation program and the predicted effects.

18. The method of claim 10, further comprising:
providing the generated rehabilitation model to a user.

19. An apparatus for supporting rehabilitation of a brain-damaged patient, the apparatus comprising:
a patient information database (DB) configured to store at least one of disease information and rehabilitation information of brain-damaged patients;
a knowledge information DB configured to store brain-related knowledge information; and
a network model generator configured to generate a brain area network model by analyzing association between brain areas based on information stored in either the patient information DB or the knowledge information DB.

20. The apparatus of claim 19, wherein the network model generator is further configured to generate the brain area network model by determining a substitute area or a compensating area of each brain area and constructing a network between a specific brain area and the determined area.

21. The apparatus of claim 20, wherein the network model generator is further configured to:
determine one or more areas capable of substituting or compensating for one or more functions of each brain area, and generate one or more sub network models for the one or more functions of each brain area; and
generate the brain area network model including the one or more generated sub network model.

22. The apparatus of claim 19, further comprising:
a rehabilitation efficiency model generator configured to generate a rehabilitation efficiency model by analyzing effects of a rehabilitation program for each brain area based on one or more information items stored in the patient information DB and the knowledge information DB.

23. The apparatus of claim 22, wherein the effects of the rehabilitation program comprises one or more of the following: a degree of functional recovery expected by applying the rehabilitation program; a participation of the patient about the rehabilitation program; a satisfaction of the patient about the rehabilitation program, a convenience of the rehabilitation program; and a psychological and a physical stress that the patient suffers during an application of the rehabilitation program.

24. The apparatus of claim 19, further comprising:
a patient information collector configured to collect the patient's rehabilitation information obtained by applying the rehabilitation model to the patient, and to store the collected rehabilitation information in the patient information DB.

25. An apparatus for supporting rehabilitation of a brain-damaged patient, the apparatus comprising:
a modeler configured to generate a brain area network model including association information of brain areas and a rehabilitation model for each brain area by analyzing eat least one of brain-related patient information and brain-related knowledge information; and
an applier configured to, in response to receipt of brain-related information of a new patient, generate a rehabilitation model to be applied to the new patient, by analyzing the brain-related information, the brain area network model, and the rehabilitation model all together.

26. The apparatus of claim 25, wherein the modeler is further configured to generate one or more sub networks for one or more functions of each brain area, and generate the brain area network model including the one or more generated sub networks.

27. The apparatus of claim 25, wherein the modeler is further configured to:
predict effects of the rehabilitation program, which includes a degree of functional recovery expected by applying the rehabilitation program to each brain area; and
generate the rehabilitation efficiency model including the predicted effects of the rehabilitation program.

28. The apparatus of claim 25, wherein the applier is further configured to:
collect the patient's rehabilitation information obtained by applying the rehabilitation model to the patient; and
transmit a feedback on the collected rehabilitation information to the modeler.
